(19) United States
(12) Patent Application Publication
(10) Pub. No.: US 2024/0200055 A1
(43) Pub. Date: Jun. 20, 2024

(54) TRANSFORMED CELL LINES EXPRESSING IL24-STAT3 AND METHODS FOR SCREENING AGENTS FOR TREATING BY TARGETING IL24-STAT3 SIGNALING PATHWAY USING THE SAME

(71) Applicant: UIF (University Industry Foundation), Yonsei University, Seoul (KR)

(72) Inventors: Jinu LEE, Seoul (KR); Sei Mee Yoon, Incheon (KR)

(73) Assignee: UIF (University Industry Foundation), Yonsei University, Seoul (KR)

(21) Appl. No.: 18/545,625

(22) Filed: Dec. 19, 2023

(30) Foreign Application Priority Data
Dec. 20, 2022 (KR) 10-2022-0179706

(51) Int. Cl. C12N 15/10; C12N 5/09; C12N 15/85

(52) U.S. Cl. CPC C12N 15/1055; C12N 5/0693; C12N 15/85; C12N 2800/107

(57) ABSTRACT

The present disclosure relates to a transformed cell line expressing the IL24-STAT3 signaling system and a method for screening a substance targeting the IL24-STAT3 signaling pathway using the same. The present disclosure provides a method for screening an inhibitor involved in the -STAT3 signaling by expressing an IL20 receptor complex binding specifically to IL24 in the presence of IL24 and activating the STAT3 signaling process.

### CROSS-REFERENCE TO RELATED APPLICATION

This application claims priority to Korean Patent Application No. 10-2022-0179706 filed on Dec. 20, 2022, and all the benefits accruing therefrom under 35 U.S.C. § 119, the contents of which in its entirety are herein incorporated by reference.

### SEQUENCE LISTING

The instant application contains a Sequence Listing which has been submitted electronically in a computer readable Sequence Listing XML format and is hereby incorporated by reference in its entirety. Said computer readable Sequence Listing in XML format was created on Dec. 18, 2023, is named G1035-26701_SequenceListing.xml and is 15,668 bytes in size.

### BACKGROUND

### 1. Field

The present disclosure relates to a transformed cell line expressing the IL24-STAT3 signaling system and method for screening a substance targeting the IL24-STAT3 signaling pathway using the same.

### 2. Description of the Related Art

Interleukin 24 (IL-24) is an important pleiotropic immunoregulatory cytokine, whose gene is located in human chromosome 1q32-33. IL-24 belongs to the IL-10 family of cytokines consisting of 9 related molecules such as IL-10, IL-19, IL-20, IL-22, IL-24, IL-26, IL-28A, IL-28B and IL-29. It is known that IL-24 induces cancer-specific cell death and is involved in the pathogenesis of allergic diseases, allergic pulmonary diseases and skin diseases as well as autoimmune diseases such as psoriasis, rheumatoid arthritis, spondyloarthropathy and inflammatory intestinal disease.

To carry out the functions described above, in response to stimulation by LPS, concanavalin A or cytokines, IL-24 signals through two types of membrane receptors (IL-22R1/IL-20R2 and IL-20R1/IL-20R2), thereby activating JAK (Janus kinase)/STAT (signal transducer and activator of transcription) signaling pathway in their cytoplasmic domains.

The activation of JAK signaling and STAT1 and STAT3 signaling pathways is induced by binding of IL-24 to the IL-20 receptor complex and it is thought that IL-20RB, IL-20RA and IL-22RA1 are involved. The IL-20RB, IL-20RA and IL-22RA1 receptors are expressed in specific tissues. IL-22RA1 is highly expressed in the skin, pancreas, liver, kidneys and intestines, and IL-20RA and IL-20RB are known to be highly expressed in the skin, lungs, ovary, testes and placenta.

It is anticipated that substances related with the IL-24 and STAT3 signaling pathway mechanisms will enable novel avenues for therapeutic intervention in both in cancer and inflammatory disease. However, the current researches merely compare the expression level of related genes through protein assay and there is no research on a cell model for studying the IL24-STAT3 signaling mechanism.

The inventors of the present disclosure have made efforts to develop a cell model for establishing a cell-based HTS system for evaluation of substances related with the signaling mechanism of IL24-STAT3. They have discovered transformed HeLa cell line wherein the expression of three genes is induced and analyzed the function of a substance involved in the IL24-STAT3 signaling mechanism using the same. As a result, they have identified that the substance has the activity of inhibiting IL24-STAT3 signaling and have completed the present disclosure.

Throughout the present specification, a number of papers and patent documents are referenced and their citations are indicated. The disclosures of the cited papers and patent documents are incorporated by reference in their entirety into the present specification to more clearly explain the level of the technical field to which the present disclosure belongs and the contents of the present disclosure.

### REFERENCE OF THE RELATED ART

### Non-Patent Documents

Non-patent document 1. “Mechanism of Action and Applications of Interleukin 24 in Immunotherapy”, Int J Mol Sci. 2016 Jun; 17(6): 869.

### SUMMARY

The inventors of the present disclosure have made consistent efforts to discover a cell model capable of confirming the signaling process in which IL24 binds to the IL20 receptor complex and activates STAT3. As a result, they have completed IL24-STAT3 signaling inhibitor screening strategy of the present disclosure by designing a cell model using the IL22RA1, IL20RB and STAT3 genes and identifying the IL24-STAT3 signaling inhibition mechanism in the presence of IL24.

The present disclosure is directed to providing a transformed HeLa cell for analyzing change in the IL24-STAT3 signaling pathway.

The present disclosure is also directed to providing a composition for measuring IL24-STAT3 signaling inhibition activity, which contains the transformed HeLa cell line.

The present disclosure is also directed to providing a method for screening an IL24-STAT3 signaling pathway inhibitor using the transformed cell line HeLa-IL24HTS.

Other purposes and advantages of the present disclosure will become more apparent by the following detailed description, claims and drawings.

The present disclosure provides a transformed HeLa cell for analyzing change in the IL24-STAT3 signaling pathway, which expresses IL24 receptors IL22RA1 (interleukin 22 receptor subunit alpha 1), IL20RB (interleukin 20 receptor subunit beta) and STAT3 (signal transducer and activator of transcription 3) genes.

The activation of STAT3 may be increased when IL24 is added to the transformed HeLa cell.

The transformed HeLa cell may be transformed with a first expression vector containing IL22RA1 (interleukin 22 receptor subunit alpha 1) represented by SEQ ID NO 1, a second expression vector containing IL20RB (interleukin 20 receptor subunit beta) represented by SEQ ID NO 2 and a third expression vector containing STAT3 (signal transducer and activator of transcription 3) represented by SEQ ID NO 3.

The present disclosure also provides a composition for measuring IL24-STAT3 signaling inhibition activity, which contains the transformed HeLa cell line.

The composition may further contain a cell line transformed with an expression vector containing a gene encoding firefly luciferase.

The composition may further 1-200 ng/mL of IL24.

The present disclosure also provides a method for screening an IL24 (interleukin 24)-STAT3 (signal transducer and activator of transcription 3) signaling pathway inhibitor, which includes:
a) a step of co-culturing the transformed HeLa cell line with a cell line transformed with an expression vector containing a gene encoding firefly luciferase;
b) a step of contacting the co-culture with 10-200 ng/mL of IL24 and a test substance expected to inhibit the IL24-STAT3 signaling mechanism;
c) a step of measuring the expression level of a reporter gene in the co-culture; and
d) a step of screening the test substance with the expression level of the reporter gene decreased by 40-99% as compared to a control group not treated with the test substance.

The method may further include a step of verifying the test substance screened in the step d), and
the verification step may include: e) a step of contacting a cell line transformed with an expression vector containing a gene encoding firefly luciferase with 10-200 ng/mL of IL24 and the test substance screened in the step c) and measuring the expression level of a reporter gene; and f) a step of excluding the test substance with the expression level of the reporter gene decreased by 20-99% as compared to a control group not treated with the test substance measured in the step e).

In the step a), the transformed HeLa cell line and the cell line transformed with the expression vector containing a gene encoding firefly luciferase may be mixed at a ratio of 1:1.

The features and advantages of the present disclosure may be summarized as follows:
(i) The present disclosure provides a transformed cell line HeLa-IL24HTS that can identify the IL24-STAT3 signaling mechanism by expressing an IL20 receptor complex binding specifically to IL24 and thereby inducing the activation of STAT3.
(ii) In addition, the present disclosure provides a method for screening an inhibitor involved in the IL24-STAT3 signaling mechanism by expressing an IL20 receptor complex binding specifically to IL24 in the presence of IL24 and activating the STAT3 signaling process.
(iii) The screening method of the present disclosure can be utilized for screening of a substance involved in the IL24-STAT3 signaling mechanism. A substance screened by this method exhibits the function of inhibiting the signaling process of STAT3 in the presence of IL24 and may be usefully utilized as a therapeutic agent for autoimmune disease or inflammatory diseases wherein the IL24-STAT3 signaling mechanism is involved.

### BRIEF DESCRIPTION OF THE DRAWINGS

FIG. 1 is the cleavage map of a pLVX-EF1a-IRES-Puro vector containing an EF1a promoter, IRES and the puromycin acetyl transferase gene (Puro).

FIG. 2 shows a result of measuring the activity of the STAT3 reporter in Hela cells in response to IL24.

FIG. 3 shows a result of measuring the activity of the STAT3 reporter in HaCaT cells in response to IL24.

FIG. 4 shows a result of measuring luciferase activity (luminescence) after treating 33 clones with IL24.

FIG. 5 shows a result of measuring the ratio of IL24(+)/IL24(−) for 10 clones (12, 28, 15, 32, 9, 10, 17, 20, 22 and 24) that showed superior results in FIG. 4 from among the 33 clones.

FIG. 6 shows a result of measuring the reporter activity of HeLa-IL24HTS cells treated with IL24 at different concentrations.

FIG. 7 shows a result of analyzing the reporter activity of HeLa-IL24HTS cells for analysis of the Z′ factor.

FIG. 8 shows a result of treating a IL24-STAT3 signaling system (STAT3-Luc2) in which the HeLa-IL24HTS cell line and the HeLa-EF1a-Luc2 cell line were co-cultured with IL24 and measuring the reporter activity after treating with DMSO or 1 μM Stattic.

### DETAILED DESCRIPTION

Hereinafter, the present disclosure will be described specifically.

In an aspect, the present disclosure relates to a transformed HeLa cell for analyzing change in the IL24-STAT3 signaling pathway, which expresses IL24 receptors IL22RA1 (interleukin 22 receptor subunit alpha 1), IL20RB (interleukin 20 receptor subunit beta) and STAT3 (signal transducer and activator of transcription 3) gene.

IL24 is a protein in the interleukin family, a type of cytokine signaling molecule in the immune system. In humans, this protein is encoded by the IL24 gene. IL-24 is a cytokine belonging to the IL-10 family of cytokines that signals through two heterodimeric receptors IL-20R1/IL-20R2 and IL-22R1/IL-20R2. This interleukin controls cell survival and proliferation by inducing rapid activation of the particular transcription factor called STAT3. This IL24-STAT3 signaling mechanism is known to play an important role in autoimmune disease and inflammatory diseases. Whereas IL-10 is an inhibitory cytokine that suppresses inflammation while maintaining immunomodulatory function, IL-24 is known to inhibit tumor growth, invasion, metastasis and angiogenesis and play an important role in the onset of inflammatory intestinal disease, psoriasis, cardiovascular diseases, rheumatoid arthritis, tuberculosis and viral infections.

In the present disclosure, a recombinant cell line in which three genes were expressed to have the IL24-STAT3 signaling mechanism known to be related to autoimmune disease and inflammatory diseases was prepared and was named as the “transformed cell line HeLa-IL24HTS”. The transformed cell line is a cell prepared by transforming with a vector containing SEQ ID NO 1 encoding IL22RA1, SEQ ID NO 2 encoding IL20RB and SEQ ID NO 3 encoding STAT3, which are IL24 receptors. It was newly developed in the present disclosure.

Previously, there was no measurement method for HTS for discovering a substance that acts on IL24-STAT3 signaling. Although the signaling can be measured by reporter assay (JBC 2002, 277: 47517), it cannot be used for HTS because the increase by IL24 is only about 3 times.

The inventors of the present disclosure have devised a cell model in which STAT3 is activated by IL24 as an IL20 receptor complex binding specifically to IL24 is expressed together with STAT3, and have identified that the occurrence of the IL24-STAT3 signaling mechanism can be confirmed simply by culturing monocytes or T cells that are difficult to culture in the presence of IL24 without additional treatment such as induction of inflammation and that its effect on the IL24-STAT3 signaling process can be identified more closely and accurately. Furthermore, a substance involved in IL24-STAT3 can be screened more quickly and conveniently by using the method described above.

Specifically, the transformed HeLa cell may express the IL22RA1, IL20RB and STAT3 genes and it is named ‘HeLa-IL24HTS’.

The IL22RA1 is a gene having a base sequence reprinted by SEQ ID NO 1 (interleukin 22 receptor subunit alpha 1) [Homo sapiens (human)] with the Gene ID: 58985 and encodes the IL22 receptor protein. The IL22 receptor protein binds to the IL10BR receptor protein to form the IL22 receptor. It is known to be expressed in the colon, duodenum, esophagus, gall bladder, kidney, liver, pancreas, skin, small intestine, stomach and thyroid.

The IL20RB is a gene having a base sequence reprinted by SEQ ID NO 2 (interleukin 20 receptor subunit beta) [Homo sapiens (human)] with the Gene ID: 53833. A protein encoded by the gene forms a heterodimeric receptor for interleukin-20 (IL20; MIM 605619). It is known to be expressed exclusively in the esophagus and skin.

The STAT3 is a gene having a base sequence reprinted by SEQ ID NO 3 (signal transducer and activator of transcription 3) [Homo sapiens (human)] with the Gene ID: 6774. In response to cytokines and growth factors, the STAT family member is phosphorylated by receptor-associated kinases, forms homo- or heterodimers and translocates to the cell nucleus where it acts as a transcription activator. Since a protein encoded by the gene mediates the expression of various genes in response to cell stimuli, it plays a key role in cell death and growth. It is known to be expressed in 25 cells including the colon, duodenum, esophagus, gall bladder, kidney, liver, pancreas, skin, small intestine, stomach, thyroid, etc.

In an example of the present disclosure, three genes (IL22RA1, IL20RB and STAT3) were identified through analysis of genes involved in the IL24-STAT3 signaling mechanism and HeLa-IL24HTS, which is a transformed cell line expressing the same, was prepared. Then, as a result of co-culturing the HeLa-IL24HTS with HeLa-EF1a-Luc2 in the presence of IL24, it was confirmed that the luciferase reporter activity is increased. In addition, by verifying that the luciferase reporter activity is decreased by the addition of a STAT3 inhibitor, it was confirmed that the cell line clearly expresses the IL24-STAT3 signaling mechanism. Through this, it was confirmed that a substance (inhibitor) involved in IL24-STAT3 can be identified and verified clearly (FIGS. 4-8).

Since the change in the IL24-STAT3 signaling pathway could not be identified if any of the three genes (IL22RA1, IL20RB and STAT3) were missing in the transformed HeLa cell, it is preferred that all the three genes are introduced into the cell.

The genes are not limited to the base sequences of SEQ ID NOS 1-3 and include the base sequences including functionally equivalent codons, codons that encode the same amino acids, or codons that encode biologically equivalent amino acids. Considering mutations having biologically equivalent activity, the base sequences used in the present disclosure also include the sequences exhibiting substantial identity to the specified sequences. The substantial identity means that the corresponding sequence exhibits at least 60% homology, more specifically 70% homology, further more specifically 80% homology, most specifically 90% homology, when aligned to match with the sequence of the present disclosure as much as possible and analyzed using an algorithm commonly used in the art.

The IL22RA1 (interleukin 22 receptor subunit alpha 1), IL20RB (interleukin 20 receptor subunit beta) and STAT3 (signal transducer and activator of transcription 3) genes can be introduced by a first expression vector containing IL22RA1 (interleukin 22 receptor subunit alpha 1) represented by SEQ ID NO 1, a second expression vector containing IL20RB (interleukin 20 receptor subunit beta) represented by SEQ ID NO 2 and a third expression vector containing STAT3 (signal transducer and activator of transcription 3) represented by SEQ ID NO 3, although not being limited thereto.

The term “vector” used in the present disclosure refers to an expression vector capable of expressing a target gene in a cell into which the vector has been introduced, or a gene construct containing essential regulatory elements operably linked to express the gene insert introduced within the vector.

In addition, the expression vector containing the gene may be any expression vector that allows expression in a Hela cell line. Specifically, it may be a viral vector. In a specific exemplary embodiment of the present disclosure, a pLVX-EF1a-IRES-Puro lentiviral vector may be used.

In addition, in the present disclosure, BSD (blasticidin S deaminase) may be introduced instead of the PAC protein to provide resistance to blasticidin S. Furthermore, the NPT2 gene may be introduced instead of the PAC protein to provide antibiotic resistance, although not being specially limited thereto.

After introducing a lentivirus containing the expression vector into a host cell, the strain may be selected finally by selecting the antibiotic.

In addition, the genes may be introduced by co-transfecting with the first expression vector, the second expression vector and the third expression vector. The method and condition for the transfection are not limited but may be selected adequately by those skilled in the art.

The lentivirus refers to an RNA virus characterized by a long incubation period. The lentivirus can transmit genetic information into the DNA of the host cell.

The inventors of the present disclosure have created a cell line that consistently and stably expresses IL-24 receptors and STAT3 at the same time by inserting the three genes into HeLa cells using the viral vector described above.

The transformed Hela cells may be cultured using methods widely known in the art. Specifically, the culturing may be performed continuously by a fed-batch or repeated fed-batch process.

In addition, suitable precursors may be used in a culture medium. The ingredients may be added to the culture medium appropriately in a batch, fed-batch or continuous process, although not being specially limited thereto. Basic compounds such as sodium hydroxide, potassium hydroxide and ammonia or acidic compounds such as phosphoric acid or sulfuric acid may be used appropriately to adjust the pH of the culture.

In another aspect, the present disclosure relates to a composition for measuring IL24-STAT3 signaling inhibition activity, which contains the transformed HeLa cell line.

The composition may further contain a cell line transformed with an expression vector containing a gene that encodes firefly luciferase.

The expression vector containing the gene that encodes firefly luciferase may be any expression vector that allows expression in the Hela cell line. In a specific exemplary embodiment of the present disclosure, a pLVX-EF1a-IRES-Puro lentiviral vector may be used. In addition, in the present disclosure, BSD (blasticidin S deaminase) may be introduced instead of the PAC protein to provide resistance to blasticidin S. In a reporter activity assay through co-culture with the transformed HeLa cell line, it was confirmed that a lentivirus containing the expression vector is introduced into a host cell can be usefully used to screen a chemical compound that inhibits the IL24-STAT3 signaling mechanism.

The firefly luciferase is a Luc2 gene derived from pGL4.1 and may be a gene amplified using a primer pair of SEQ ID NO 10 and SEQ ID NO 11.

In the present disclosure, a system for screening a chemical substance that inhibits the IL24-STAT3 signaling mechanism has been identified. Specifically, an inhibitor involved in the IL24-STAT3 signaling mechanism can be screened by measuring the degree of inhibited function due to binding of IL24 to the receptor or activation of STAT3 using the transformed HeLa cell line.

When the composition further contains the cell line transformed with the expression vector containing a gene that encodes firefly luciferase, it can be identified whether a chemical substance (test substance) inhibits the IL24-STAT3 signaling mechanism by verifying the background signal of the chemical substance that inhibits the IL24-STAT3 signaling mechanism through reporter activity.

The transformed HeLa cell line and the cell line transformed with the expression vector containing a gene that encodes firefly luciferase may be contained in different vials and may be used as a kit in that state.

The composition may further contain 1-200 ng/mL of IL24. When 1-200 ng/mL of IL24 is added after culturing the transformed HeLa cell line, IL24 binds to an IL-20 receptor complex expressed from the transformed HeLa cell line, thereby activating STAT3 increasing reporter activity. Therefore, when a test substance is added, it can measure the inhibition activity for the IL24-STAT3 signaling mechanism sensitively and quickly by measuring the decrease of the reporter activity.

As a result of analyzing reporter activity after adding the screened test substance together with 1-200 ng/mL of IL24 to the cell line transformed with the expression vector containing a gene that encodes firefly luciferase, if the reporter activity has decreased, the test substance may be determined as not having the ability of inhibiting IL24-STAT3 signaling.

Since the composition of the present disclosure can be applied to any application wherein the IL24-STAT3 signaling mechanism is involved, it can be applied to various applications. In addition, it is effective for ultra-fast screening analysis because the Z′ factor is 0.5 or higher.

In another aspect, the present disclosure relates to a method for screening an IL24 (interleukin 24)-STAT3 (signal transducer and activator of transcription 3) signaling pathway inhibitor, which includes:
a) a step of contacting a transformed HeLa cell line with 10-200 ng/mL of IL24 and a test substance expected to inhibit the IL24-STAT3 signaling mechanism;
b) a step of measuring the expression level of a reporter gene from a culture of the cell line; and
c) a step of screening the test substance with the expression level of the reporter gene decreased by 40-99% as compared to a control group not treated with the test substance.

First, the transformed HeLa cell line is cultured to 30-90% confluency. The description of the transformed HeLa cell line will be omitted to avoid redundancy.

Next, the transformed HeLa cell line is contacted with 10-200 ng/mL of IL24 and a test substance expected to inhibit the IL24-STAT3 signaling mechanism.

The activation of STAT3 is induced by adding 10-200 ng/mL of IL24 to the transformed HeLa cell line. The test substance is a substance expected to inhibit the IL24-STAT3 signaling mechanism and refers to a substance whose effect on the IL24-STAT3 signaling mechanism is to be analyzed. The test substance may include any molecule, e.g., an extract, a protein, an oligopeptide, a small organic molecule, a polysaccharide, a polynucleotide, etc.

Next, b) the expression level of a reporter gene is measured from a culture of the cell line of the step a). Any known method may be used for the measurement of the expression level of a reporter gene from the culture of the step a). For example, the expression of the gene may be investigated by measuring the degree of color development such as fluorescence intensity.

In the present disclosure, since the reporter gene is expressed only when STAT3 is activated from the transformed HeLa cell line, the IL24-STAT3 signaling pathway inhibition activity may be investigated by comparing the expression level of a group treated with the test substance with that of a group not treated with the test substance.

Finally, in the step c), the test substance with the expression level of the reporter gene decreased by 40-99% may be screened by comparing the measurement result in the b) with that of the control group not treated with the test substance.

In the present disclosure, the ‘control group’ also called the ‘group not treated with the test substance’ refers to a co-culture not treated with the test substance or a sample isolated therefrom, which is in a parallel relationship with the group treated with the test substance.

The method may further include a step of verifying the test substance screened in the step c).

The verification step may include: d) a step of contacting a cell line transformed with an expression vector containing a gene encoding firefly luciferase with 10-200 ng/mL of IL24 and the test substance screened in the step c) and measuring the expression level of a reporter gene; and e) a step of excluding the test substance with the expression level of the reporter gene decreased by 20-99% as compared to a control group not treated with the test substance measured in the step d).

In the present disclosure, the test substance screened by the screening may be regarded as having inhibition activity against the IL24-STAT3 signaling mechanism and may be regarded as a therapeutic agent for a disease related with IL24-STAT3 signaling. The disease related with IL24-STAT3 signaling may be cancer disease, an autoimmune disease or an inflammatory disease.

The cancer disease is not specially limited as long as it is a cancer disease related with IL24-STAT3 signaling. Specifically, it may be one or more selected from a group consisting of benign astrocytoma, malignant astrocytoma, pituitary adenoma, meningioma, cerebral lymphoma, oligodendroglioma, craniopharyngioma, ependymoma, brain stem tumor, laryngeal cancer, oropharyngeal cancer, nasal cavity/paranasal sinus cancer, nasopharyngeal cancer, salivary gland cancer, hypopharyngeal cancer, thyroid cancer, oral cancer, thoracic tumor, small cell lung cancer, non-small cell lung cancer, thymus gland cancer, mediastinal tumor, esophageal cancer, breast cancer, abdominal tumor, stomach cancer, liver cancer, gallbladder cancer, biliary tract cancer, pancreatic cancer, small intestine caner, colon cancer, anal cancer, bladder cancer, kidney cancer, penile cancer, prostate cancer, cervical cancer, endometrial tumor, ovarian cancer, uterine sarcoma, vaginal cancer, vulvar cancer and skin cancer, although not being limited thereto.

The autoimmune disease may be one or more selected from systemic lupus erythematosus, insulin-dependent diabetes, multiple sclerosis, autoimmune encephalomyelitis, rheumatoid arthritis, juvenile rheumatoid arthritis, psoriatic arthritis, discoid lupus erythematosus, photosensitivity skin disease, autoimmune arthritis, myasthenia gravis, thyroiditis, experimental form of uveitis, Hashimoto's thyroiditis, primary myxedema, thyrotoxicosis, pernicious anemia, autoimmune atrophic gastritis, Addison's disease, premature menopause, male infertility, juvenile diabetes, Goodpasture syndrome, pemphigus, bullous pemphigus, sympathetic ophthalmitis, phacogenic uveitis, autoimmune hemolytic anemia, idiopathic leukopenia, primary biliary cirrhosis, chronic active hepatitis Hbs-ve, cryptogenic cirrhosis, ulcerative colitis, Sjögren syndrome, scleroderma, Wegener's granulomatosis, polymyositis/dermatomyositis and discoid LE, although not being limited thereto.

The inflammatory disease may be one or more selected from dermatitis, allergy, atopy, asthma, conjunctivitis, periodontitis, rhinitis, otitis media, pharyngitis, tonsillitis, pneumonia, gastric ulcer, gastritis, Crohn's disease, colitis, gout, ankylosing spondylitis, lupus, fibromyalgia, psoriatic arthritis, osteoarthritis, rheumatoid arthritis, frozen shoulder, tendinitis, tenosynovitis, peritendinitis, myositis, hepatitis, cystitis, nephritis, Sjögren syndrome, multiple sclerosis and acute and chronic inflammatory diseases. Specifically, it may be a chronic inflammatory disease. Specific examples of the chronic inflammatory disease include psoriasis, psoriatic arthritis, rheumatoid arthritis, multiple sclerosis, systemic lupus erythematosus, inflammatory intestinal disease, Crohn's disease, ulcerative colitis, ankylosing spondylitis, asthma, chronic obstructive pulmonary disease, periodontitis and idiopathic inflammatory myopathy, although not being limited thereto.

Hereinafter, the present disclosure will be described in more detail through examples. The examples are solely for explaining the present disclosure more specifically and it will be obvious to those having ordinary knowledge in the art that the scope of the present disclosure is not limited by the examples.

### Experimental methods

### Construction of pLVX-EIBla

A pLVX-EF1a-IRES-Puro vector (hereinafter, “pLVX-EIP”) including an EF1a promoter, IRES and the puromycin acetyl transferase gene (Puro) was purchased from Clontech. “pLVX-EIBla” was constructed by exchanging the PAC (puromycin acetyl transferase) gene of the plasmid with BSD (blasticidin S deaminase).

### Construction of pLVX-EIN

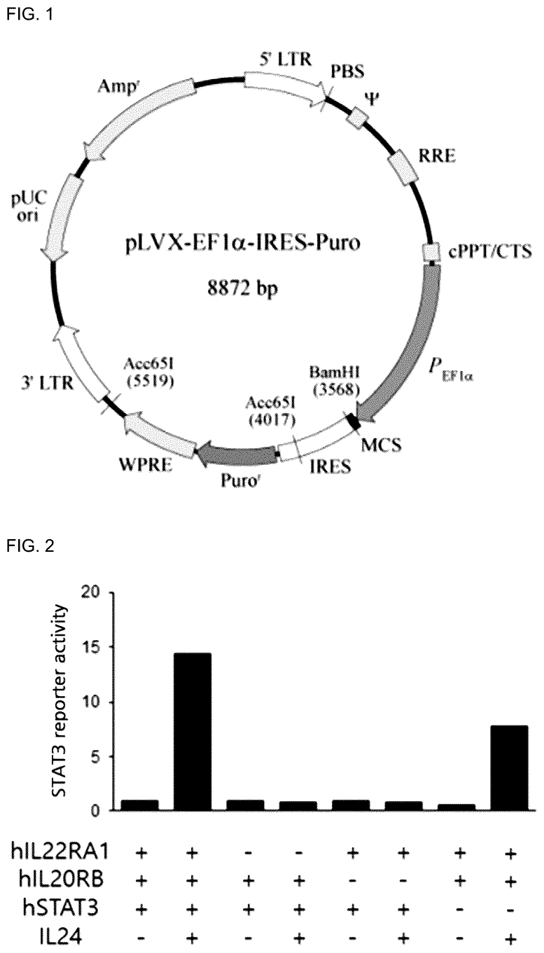

A pLVX-EF1a-IRES-Puro vector (hereinafter, “pLVX-EIP”) including an EF1a promoter, IRES and the puromycin acetyl transferase gene (Puro) was purchased from Clontech (Cat No. 631988) (FIG. 1). “pLVX-EIN” was constructed by exchanging the PAC (puromycin acetyl transferase) gene of the plasmid with NPT2 (neomycin phosphotransferase 2).

### Construction of pLVX-EIP-hIL22RA1

The coding region of pCMV-SPORT6-hIL22RA1 (Korea Human Gene Bank) (original clone no. IRAK-52-B11) was amplified by PCR using 5′-ATA TAC AAT TGC CAC CAT GAG GAC GCT GCT GA-3′ (SEQ ID NO 4) and 5′-TAT ATG CTA GCT CAG GAC TCC CAC TGC-3′ (SEQ ID NO 5) as a primer pair. After obtaining an hIL22RA1 fragment by cleaving the same using the Mfel and Nhel restriction enzymes, it was inserted into the EcoRI/XbaI site of the pLVX-EIP vector (Clontech) to construct pLVX-EIP-hIL22RA1.

### Construction of pLVX-EIBla-hIL20RB

The coding region of pCMV3-hIL20RB (Sinobiological Cat: HG29609-UT) was amplified by PCR using 5′-ATA TAC GTC TCA AAT TAC CAC AAT GCA GAC TTT CAC AAT GG-3′ (SEQ ID NO 6) and 5′-ATA TAC GTC TCT CTA GAC TAT GAG ATC CAG GCC-3′ (SEQ ID NO 7) as a primer pair. After obtaining an hIL20RB fragment by cleaving the same using the BsmBI restriction enzyme, it was inserted into the EcoRI/XbaI site of the pLVX-EIBla vector to construct pLVX-EIBla-hIL20RB.

### Construction of pLVX-EIN-hSTAT3

The coding region of pCMV3-hSTAT3 (Sinobiological Cat: HG10034-UT) was amplified by PCR using 5′-ATA TAC GTC TCA AAT TAC AAC AAT GGC CCA ATG GAA TCA-3′ (SEQ ID NO 8) and 5′-ATA TAC GTC TCA CTA GAT CAC ATG GGG GAG GTA-3′ (SEQ ID NO 9) as a primer pair. After obtaining an hSTAT3 fragment by cleaving the same using the BsmBI restriction enzyme, it was inserted into the EcoRI/XbaI site of the pLVX-EIN vector to construct pLVX-EIN-hSTAT3.

### Construction of pLVX-EIP-Luc2

The coding region of pGL4.1 (Promega, E6651) was amplified by PCR using 5′-TAT ATG AAT TCG CCA CCA TGG AAG ATG C-3′ (SEQ ID NO 10) and 5′-TAT ATT CTA GAA TTA CAC GGC GAT CTT GC-3′ (SEQ ID NO 11) as a primer pair. After obtaining a Luc2 fragment by cleaving the same using the EcoRI/XbaI restriction enzymes, it was inserted into the EcoRI/XbaI site of the pLVX-EIP vector to construct pLVX-EIP-Luc2.

### Production and Processing of Lentivirus Expressing hIL22RA1, hIL20RB, hSTAT3 or Luc2

The transduction of “pLVX-EIP-hIL22RA1”, “pLVX-EIBla-hIL20RB”, “pLVX-EIN-hSTAT3” and “pLVX-EIP-Luc2” was performed as follows.

One of the four plasmids prepared above (“pLVX-EIP-hIL22RA1”, “pLVX-EIBla-hIL20RB”, “pLVX-EIN-hSTAT3” and “pLVX-EIP-Luc2”) was mixed with psPAX2 (Addgene #12260) and pMD2.G (Addgene #12259) at a rate of 4:3:1 and co-transfected into 293T cells, which are human embryonic kidney cells. The transfected cells were cultured for 48 hours in a 37° C. incubator. To obtain secreted lentivirus after the culturing, the culture was centrifuged at 1000 rpm for 5 minutes and the supernatant was passed through a 0.45-μm syringe filter.

For lentivirus treatment, Hela cells were cultured to 20% confluency on the day of infection. The lentivirus was mixed with a culture medium at 1:1 and then treated to the cells. After adding 2 μg of Polybrene per 1 mL of the virus, the plate was carefully shaken for mixing. After culturing for 15 hours under the condition of 37° C. and 5% CO₂, the culture medium was replaced with a fresh one. 48 hours later, the transduced cells were cultured further and recombinant cells were selected by adding an antibiotic to the medium.

### Establishment of HeLa-4.47 Cell Line

2 mg of pGL4.47 (Promega, E4041), which is a STAT3 reporter plasmid, was linearized using the Pst1 restriction enzyme. The linearized plasmid was transfected into Hela cells using polyethyleneimine (Polysciences). 48 hours later, the cells were transferred to a 100-mm culture dish and only the transfected cells were selected by treating with 500 μg/mL hygromycin and culturing for 2 weeks. The selected transfected cells were cultured on a 96-well plate. The cells were dispensed at 0.5 cell per well. 33 clones were obtained by culturing further for 3 weeks. Each clone was mixed with pCMV-SPORT6-hIL22RA1 (Korea Human Gene Bank), pCMV3-hIL20RB (SinoBiological) and pCMV3-hSTAT3 (SinoBiological) at 1:1:1 for co-transfection. The transfected cells were treated or untreated with 150 ng/mL IL24. After culturing for 24 hours, luciferase activity was measured by luciferase assay. The clone with the strongest activity was established and named HeLa-4.47.

### Establishment of HeLa-IL24HTS Cell Line

The selected and established HeLa-4.47 cells were cultured to 20% confluency on the day of infection. After removing the culture medium, lentivirus expressing hIL22RA1, hIL20RB or hSTAT3 was mixed with a culture medium and treated to the cells. After adding 2 μg of Polybrene per 1 mL of the virus, the plate was carefully shaken for mixing. After culturing for 15 hours under the condition of 37° C. and 5% CO₂, the culture medium was replaced with a fresh one. 48 hours later, the transduced cells were cultured further and recombinant HeLa-IL24HTS cells were selected by culturing for 2 weeks in a medium containing 2 μg/mL puromycin, 5 μg/mL blasticidin S and 1,000 μg/mL G418 as antibiotics. Through this experiment, HeLa-4.47-hIL22RA1-hIL20RB-hSTAT3 cells that stably express all of hIL22RA1, hIL20RB and hSTAT3 were established and named HeLa-IL24HTS.

### Establishment of HeLa-EF1a-Luc2 Cell Line

Hela cells were cultured to 20% confluency on the day of infection. After removing the culture medium, lentivirus expressing Luc2 (LVX-EIP-Luc2) was mixed with a culture medium and treated to the cells. After adding 2 μg of Polybrene per 1 mL of the virus, the plate was carefully shaken for mixing. After culturing for 15 hours under the condition of 37° C. and 5% CO₂, the culture medium was replaced with a fresh one. 48 hours later, the transduced cells were cultured further and recombinant HeLa-EF1a-Luc2 cells were selected by culturing for 2 weeks in a medium containing 2 μg/mL puromycin as an antibiotic.

### Measurement of Z′ Factor

The “HeLa-IL24HTS” cells were cultured to 50% confluency on a 96-well plate. 20 wells were treated with 150 ng/mL IL24 and 20 wells were treated with a medium only. After culturing for 24 hours, luciferase activity was measured by luciferase assay and Z′ factor was calculated according to Equation 1.

$${Z}^{′} factor = 1 - 3 \times ( IL 24 ( + ) σ + IL 24 ( - ) σ ) / ( IL 24 ( + ) μ - IL 24 ( - ) μ ) \qquad [ Equation 1 ]$$

In Equation 1, σ represents standard deviation and μ represents arithmetic mean.

### <TEST EXAMPLE 1> DETERMINATION OF GENES NECESSARY FOR CELLS FOR MEASURING IL24-STAT3 SIGNALING

In order to identify the genes required for verification of IL24-STAT3 signaling, HeLa and transformed HaCaT cells were prepared performing transient transfection and luciferase activity was measured using a luciferase assay system (Promega) with or without IL24 treatment.

First, the HeLa or HaCaT cells were dispensed on a 24-well plate and cultured to 50% confluency. The cells were co-transfected by treating with 500 ng of “pCMV-hIL22RA1”, “pCMV3-hIL20RB”, “pCMV3-hSTAT3”, “pGL4.47” or a mixture thereof. After culturing for 24 hours, the transfected cells were treated or untreated with 150 ng/mL IL24. After culturing for 24 hours, luciferase activity was measured by luciferase assay. The presence or absence of the treatment was marked as +or −, respectively.

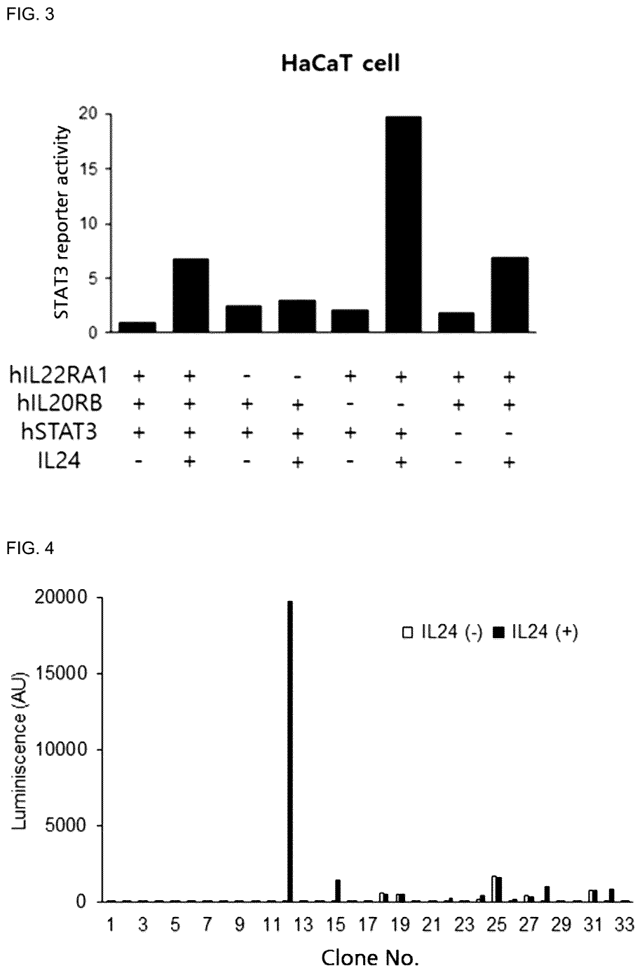

FIG. 2 shows a result of measuring the activity of the STAT3 reporter in the HeLa cells in response to IL24, and FIG. 3 shows a result of measuring the activity of the STAT3 reporter in the HaCaT cells in response to IL24.

In this test example, the verification of IL24-STAT3 signaling was analyzed according to the expression of the three genes (IL22RA1, IL20RB and STAT3) selected for the measurement of the activity of the IL24-STAT3 pathway in the Hela and HaCaT cells.

As shown in FIGS. 2 and 3, the HaCaT cells responded the most highly to the IL24-STAT3 signaling in response to IL24 when the IL20RB gene was not expressed and only IL22RA1 and STAT3 were expressed. And, the HeLa cells responded the most highly when all of IL22RA1, IL20RB and STAT3 were expressed. Therefore, it can be seen that the genes to be expressed vary depending on the cells.

Hela cells are advantageous for screening because they grow quickly and adhere strongly to the 96-well plate. For the Hela cells, the porter activity was induced best when all of the IL22RA1, IL20RB and STAT3 genes were expressed, suggesting that it is desirable that all the three genes are expressed in the cells.

### <TEST EXAMPLE 2> SELECTION OF HeLa-4.47 CELL LINE AND ESTABLISHMENT OF HeLa-IL24HTS

HeLa cells were transfected with pGL4.47 and selecting using hygromycin to obtain 33 clones. After co-transfecting each clone by mixing with pCMV-SPORT6-hIL22RA1, pCMV3-hIL20RB and pCMV3-hSTAT3 at 1:1:1, luciferase activity was measured using a luciferase assay system (Promega) for treatment with 150 ng/mL IL24 (IL24(+)) or absence of treatment (IL24(−)).

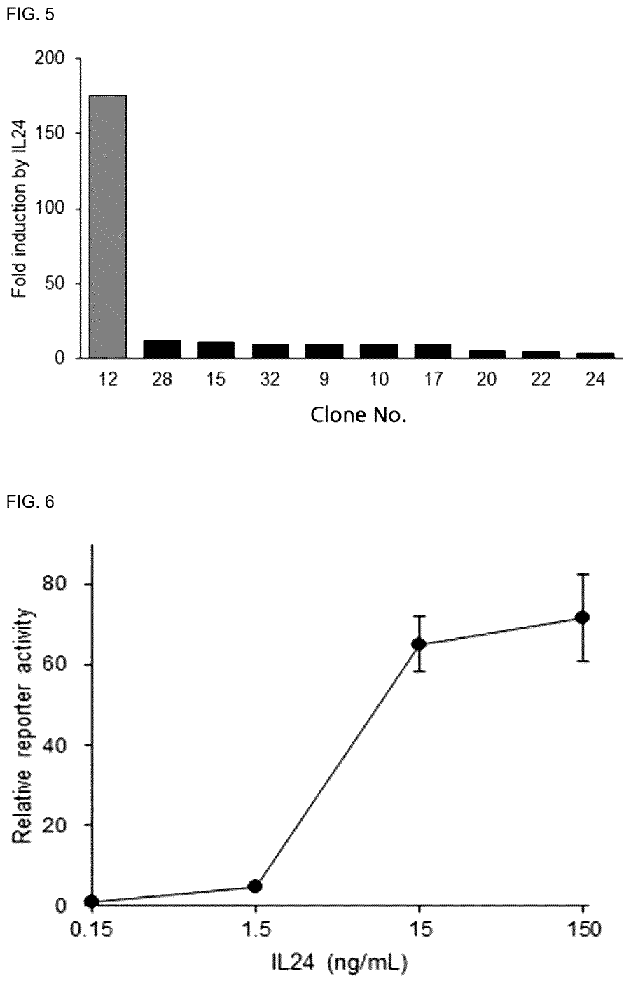

FIG. 4 shows a result of measuring luciferase activity (luminescence) after treating the 33 clones with IL24, FIG. 5 shows a result of measuring the ratio of IL24(+)/IL24(−) for 10 clones (12, 28, 15, 32, 9, 10, 17, 20, 22 and 24) that showed superior results in FIG. 4 from among the 33 clones.

As shown in FIG. 4 and FIG. 5, among the 33 clones obtained by transfecting the Hela cells with pGL4.47 and selecting with hygromycin, the clone 12 showed the highest inducibility. The cells that showed the strongest activity for IL24 treatment, with the IL24(+)/IL24(−) ratio of 150-200, were selected and established as HeLa-4.47. The selected HeLa-4.47 cell line also showed the highest fold induction by IL24.

Then, the selected HeLa-4.47 cells were treated with lentivirus to establish HeLa-IL24HTS cells which stably express all of hIL22RA1, hIL20RB and hSTAT3.

### <TEST EXAMPLE 3> CONFIRMATION OF EXPRESSION IN HeLa-IL24HTS CELLS DEPENDING ON TREATMENT WITH IL24 AT DIFFERENT CONCENTRATIONS

The HeLa-4.47 cells established in Test Example 2 were treated with lentivirus expressing hIL22RA1, hIL20RB or hSTAT3 and selected with 2 μg/mL puromycin, 5 μg/mL blasticidin S and 1,000 μg/mL G418 as antibiotics to obtain HeLa-4.47-hIL22RA1-hIL20RB-hSTAT3 cells (HeLa-IL24HTS) which stably express all of hIL22RA1, hIL20RB and hSTAT3. The specific process of preparing the HeLa-IL24HTS cell line was the same as described above.

After treating the prepared HeLa-IL24HTS cells with IL24 at different concentrations (0.15 ng/mL, 1.5 ng/mL, 15 ng/mL and 150 ng/mL) and culturing for 24 hours, luciferase activity was measured using a luciferase assay system (Promega). FIG. 6 shows a result of measuring the reporter activity of the HeLa-IL24HTS cells treated with IL24 at different concentrations. It can be seen that the HeLa-IL24HTS cells show strong reporter induction of 60 fold or higher in the presence of 15 ng/mL or higher IL24.

### <TEST EXAMPLE 4> APPLICABILITY HeLa-IL24HTS CELLS FOR HTS

After treating the HeLa-IL24HTS cells with 150 ng/mL IL24 (IL24(+)) or without treating (IL24(−)) and culturing the cells for 24 hours, luciferase activity was measured using a luciferase assay system (Promega).

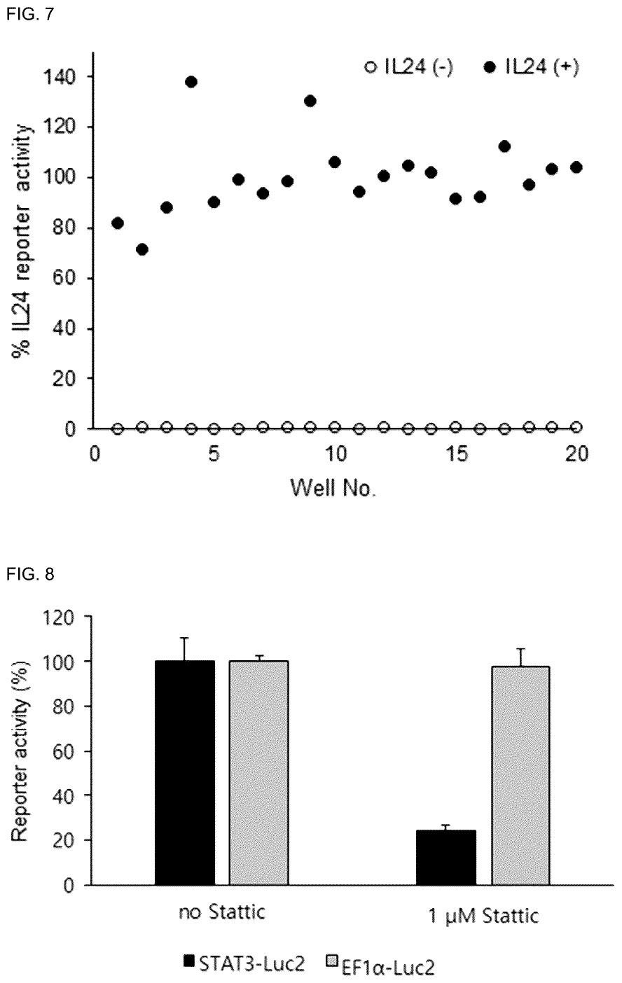

FIG. 7 shows a result of analyzing the reporter activity of the HeLa-IL24HTS cells for analysis of the Z′ factor. The Z′ factor calculated form FIG. 7 was 0.54. Since the HeLa-IL24HTS cells according to the present disclosure showed strong reporter induction when treated with IL24, Z′ factor was measured to evaluate whether they can be used for HTS. Since the Z′ factor was measured as 0.54, it can be seen that the cells are suitable for HTS.

### <TEST EXAMPLE 5> CONFIRMATION OF EFFECT OF KNOWN IL24-STAT3 SIGNALING INHIBITOR IN HeLa-IL24HTS CELLS

HeLa-IL24HTS cells and HeLa-EF1a-Luc2 cells were cultured on a 96-well plate at a concentration of 2.0×10⁴ cells/well, respectively. After treating the cells with a vehicle (DMSO) (no Stattic) or 1 μM Stattic (CAS No: 19983-44-9), which is a STAT3 inhibitor, while treating with 150 ng/mL IL24 and culturing for 8 hours, luciferase activity was measured using a luciferase assay system (Promega).

FIG. 8 shows a result of treating the HeLa-IL24HTS cells or HeLa-EF1a-Luc2 cells with DMSO or 1 μM Stattic while treating with IL24 and measuring reporter activity.

As shown in FIG. 8, when the cells were treated with Stattic, which is a STAT3 inhibitor, reporter activity was inhibited by about 80% for the HeLa-IL24HTS cells but no significant change was observed for the HeLa-EF1a-Luc2 cells, suggesting that the HeLa-IL24HTS cells according to the present disclosure can screen the substance related with IL24-STAT3 signaling accurately and quickly.

Through the above results, it was confirmed that the HeLa-IL24HTS cell line according to the present disclosure has an inhibitory effect against the activity of Stattic, which is known as an IL24-STAT3 signaling inhibitor. Therefore, a screening method using the HeLa-IL24HTS cells prepared in the present disclosure can be utilized for fast high-throughput screening of a test substance for regulation of IL24-STAT3 signaling.

While specific exemplary embodiments of the present disclosure have been described in detail, it will be obvious to those having ordinary knowledge in the art that they are merely specific exemplary embodiments and the scope of the present disclosure is not limited by them. Therefore, the substantial scope of the present disclosure is defined by the appended claims and their equivalents.

What is claimed is:

1. A transformed HeLa cell for analyzing change in the IL24-STAT3 signaling pathway, which expresses IL24 receptors IL22RA1 (interleukin 22 receptor subunit alpha 1), IL20RB (interleukin 20 receptor subunit beta) and STAT3 (signal transducer and activator of transcription 3) genes.

2. The transformed HeLa cell according to claim 1, wherein the activation of STAT3 is increased when IL24 is added.

3. The transformed HeLa cell according to claim 1, wherein the transformed HeLa cell is transformed with a first expression vector comprising IL22RA1 (interleukin 22 receptor subunit alpha 1) represented by SEQ ID NO 1, a second expression vector comprising IL20RB (interleukin 20 receptor subunit beta) represented by SEQ ID NO 2 and a third expression vector comprising STAT3 (signal transducer and activator of transcription 3) represented by SEQ ID NO 3.

4. A composition for measuring IL24-STAT3 signaling inhibition activity, comprising the transformed HeLa cell line according to claim 1.

5. The composition for measuring IL24-STAT3 signaling inhibition activity according to claim 4, wherein the composition further comprises a cell line transformed with an expression vector comprising a gene encoding firefly luciferase.

6. The composition for measuring IL24-STAT3 signaling inhibition activity according to claim 4, wherein the composition further comprises 1-200 ng/mL of IL24.

7. A method for screening an IL24 (interleukin 24)-STAT3 (signal transducer and activator of transcription 3) signaling pathway inhibitor, comprising:
a) a step of contacting the transformed HeLa cell line according to claim 1 with 10-200 ng/mL of IL24 and a test substance expected to inhibit the IL24-STAT3 signaling mechanism;
b) a step of measuring the expression level of a reporter gene from a culture of the cell line; and
c) a step of screening the test substance with the expression level of the reporter gene decreased by 40-99% as compared to a control group not treated with the test substance.

8. The method for screening an IL24-STAT3 signaling pathway inhibitor according to claim 7, which further comprises a step of verifying the test substance screened in the step c),
wherein the verification step comprises:
d) a step of contacting a cell line transformed with an expression vector comprising a gene encoding firefly luciferase with 10-200 ng/mL of IL24 and the test substance screened in the step c) and measuring the expression level of a reporter gene; and
e) a step of excluding the test substance with the expression level of the reporter gene decreased as compared to a control group not treated with the test substance measured in the step d).


SEQUENCE LISTING (11 sequences)

SEQ ID NO: 1 - DNA, 1725 bp, Homo sapiens
  source 1..1725: mol_type = genomic DNA
atgaggacgctgctgaccatcttgactgtgggatccctggctgctcacgcccctgaggacccctcggatctgctccagcacgtgaaattccagtccagcaactttgaaaacatcctgacgtgggacagcgggccggagggcaccccagacacggtctacagcatcgagtataagacgtacggagagagggactgggtggcaaagaagggctgtcagcggatcacccggaagtcctgcaacctgacggtggagacgggcaacctcacggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacatggacctactccttctccggagccttcctgttctccatgggcttcctcgtcgcagtactctgctacctgagctacagatatgtcaccaagccgcctgcacctcccaactccctgaacgtccagcgagtcctgactttccagccgctgcgcttcatccaggagcacgtcctgatccctgtctttgacctcagcggccccagcagtctggcccagcctgtccagtactcccagatcagggtgtctggacccagggagcccgcaggagctccacagcggcatagcctgtccgagatcacctacttagggcagccagacatctccatcctccagccctccaacgtgccacctccccagatcctctccccactgtcctatgccccaaacgctgcccctgaggtcgggcccccatcctatgcacctcaggtgacccccgaagctcaattcccattctacgccccacaggccatctctaaggtccagccttcctcctatgcccctcaagccactccggacagctggcctccctcctatggggtatgcatggaaggttctggcaaagactcccccactgggacactttctagtcctaaacaccttaggcctaaaggtcagcttcagaaagagccaccagctggaagctgcatgttaggtggcctttctctgcaggaggtgacctccttggctatggaggaatcccaagaagcaaaatcattgcaccagcccctggggatttgcacagacagaacatctgacccaaatgtgctacacagtggggaggaagggacaccacagtacctaaagggccagctccccctcctctcctcagtccagatcgagggccaccccatgtccctccctttgcaacctccttcccgtccatgttccccctcggaccaaggtccaagtccctggggcctgctggagtcccttgtgtgtcccaaggatgaagccaagagcccagcccctgagacctcagacctggagcagcccacagaactggattctcttttcagaggcctggccctgactgtgcagtgggagtcctga

SEQ ID NO: 2 - DNA, 936 bp, Homo sapiens
  source 1..936: mol_type = genomic DNA
atgcagactttcacaatggttctagaagaaatctggacaagtcttttcatgtggtttttctacgcattgattccatgtttgctcacagatgaagtggccattctgcctgcccctcagaacctctctgtactctcaaccaacatgaagcatctcttgatgtggagcccagtgatcgcgcctggagaaacagtgtactattctgtcgaataccagggggagtacgagagcctgtacacgagccacatctggatccccagcagctggtgctcactcactgaaggtcctgagtgtgatgtcactgatgacatcacggccactgtgccatacaaccttcgtgtcagggccacattgggctcacagacctcagcctggagcatcctgaagcatccctttaatagaaactcaaccatccttacccgacctgggatggagatcaccaaagatggcttccacctggttattgagctggaggacctggggccccagtttgagttccttgtggcctactggaggagggagcctggtgccgaggaacatgtcaaaatggtgaggagtgggggtattccagtgcacctagaaaccatggagccaggggctgcatactgtgtgaaggcccagacattcgtgaaggccattgggaggtacagcgccttcagccagacagaatgtgtggaggtgcaaggagaggccattcccctggtactggccctgtttgcctttgttggcttcatgctgatccttgtggtcgtgccactgttcgtctggaaaatgggccggctgctccagtactcctgttgccccgtggtggtcctcccagacaccttgaaaataaccaattcaccccagaagttaatcagctgcagaagggaggaggtggatgcctgtgccacggctgtgatgtctcctgaggaactcctcagggcctggatctcatag

SEQ ID NO: 3 - DNA, 2313 bp, Homo sapiens
  source 1..2313: mol_type = genomic DNA
atggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtga

SEQ ID NO: 4 - DNA, 32 bp, synthetic construct
  source 1..32: mol_type = other DNA
atatacaattgccaccatgaggacgctgctga

SEQ ID NO: 5 - DNA, 27 bp, synthetic construct
  source 1..27: mol_type = other DNA
tatatgctagctcaggactcccactgc

SEQ ID NO: 6 - DNA, 41 bp, synthetic construct
  source 1..41: mol_type = other DNA
atatacgtctcaaattaccacaatgcagactttcacaatgg

SEQ ID NO: 7 - DNA, 33 bp, synthetic construct
  source 1..33: mol_type = other DNA
atatacgtctctctagactatgagatccaggcc

SEQ ID NO: 8 - DNA, 39 bp, synthetic construct
  source 1..39: mol_type = other DNA
atatacgtctcaaattacaacaatggcccaatggaatca

SEQ ID NO: 9 - DNA, 33 bp, synthetic construct
  source 1..33: mol_type = other DNA
atatacgtctcactagatcacatgggggaggta

SEQ ID NO: 10 - DNA, 28 bp, synthetic construct
  source 1..28: mol_type = other DNA
tatatgaattcgccaccatggaagatgc

SEQ ID NO: 11 - DNA, 29 bp, synthetic construct
  source 1..29: mol_type = other DNA
tatattctagaattacacggcgatcttgc